(19) United States
(12) Patent Application Publication
(10) Pub. No.: US 2014/0072541 A1
(43) Pub. Date: Mar. 13, 2014

(54) PROBIOTIC FUNCTIONAL FOOD SUITABLE FOR IMMUNOCOMPROMISED INDIVIDUALS UNDERGOING TREATMENT SUCH AS CHEMOTHERAPY AND/OR RADIOTHERAPY

(71) Applicants: Maria Loreto Ormeno Saavedra, Concepcion (CL); Erica Castro Inostroza, Concepcion (CL); Rodrigo Borquez Yanez, Concepcion (CL); Margarita Gonzalez Riquelme, Concepcion (CL); Rodrigo Vera Garcia, Concepcion (CL); Natalia Toledo Aguilar, Concepcion (CL)

(72) Inventors: Maria Loreto Ormeno Saavedra, Concepcion (CL); Erica Castro Inostroza, Concepcion (CL); Rodrigo Borquez Yanez, Concepcion (CL); Margarita Gonzalez Riquelme, Concepcion (CL); Rodrigo Vera Garcia, Concepcion (CL); Natalia Toledo Aguilar, Concepcion (CL)

(73) Assignees: UNIVERSIDAD DE CONCEPCION, Concepcion (CL); Maria Loreto ORMENO SAAVEDRA, Concepcion (CL)

(21) Appl. No.: 13/879,611

(22) PCT Filed: Oct. 14, 2011
(86) PCT No.: PCT/CL2011/000064

§ 371 Date: Oct. 4, 2013

(30) Foreign Application Priority Data
Oct. 14, 2010 (CL) 1124-2010

(51) Int. Cl. A61K 35/74; A23G 9/36

(52) U.S. Cl. CPC A61K 35/747; A23G 9/363
USPC 424/93.45

(57) ABSTRACT

The invention relates to a probiotic functional food comprising: a biomass of Lactobacillus spp. strains conditioned to withstand temperatures of between −30 and −40° C., and cryoprotective agents. Said food comprises jellies, desserts, juices and/or milk derivatives, preferably ice cream. This functional food is intended for patients undergoing therapies such as chemotherapy and/or radiotherapy, since it counteracts the side effects of the cancer treatment.

### STATE OF THE ART

Over the last few years, interest in preventive therapies and nutritional supplements to improve health has been increasing. The fact that human beings are genetically predisposed to acquire certain diseases has led researchers to look for new alternatives to prevent these diseases, among which diet is one of the most important factors. According to the American Cancer Society, 1.5 million new cancer cases are registered each year and it is estimated that 70% of these cases are due to diet. The devastating effects of this disease can be prevented or attenuated by a balanced diet, with a consequent change in eating habits.

In Chile, every year about 40,000 individuals develop some type of cancer. However, a high percentage of cancer sufferers in the world now live with the disease or have recovered from it through surgery and follow-up treatment with chemotherapy and/or radiotherapy.

The main goal of this type of treatment is to cure the disease, but some types of cancer cannot be cured, in which case therapies are used to improve the quality of life of the patient in some way.

The drugs used in chemotherapy do not only affect cancer cells but also normal cells and they generally have undesirable side effects.

Nausea, vomiting, fatigue, hair loss and severe stomach problems, in addition to symptoms that are imperceptible to the patient, such as a fall in red and white blood cell counts and blood platelets, are some of the side effects of chemotherapy about which patients frequently complain.

Most of these effects disappear when treatment ends, enabling healthy cells to stabilize their metabolism and reproduce to reach their normal number. In the meantime, there are various ways in which patients can control particular minor problems. In the case of severe side effects, the doctor makes certain recommendations to palliate or relieve unpleasant reactions to some extent.

In general, people tend to feel better during cancer treatment if they rest enough and have a balanced diet. Many patients need to sleep more, as well as to ingest more proteins and calories, to help the body to recover. Chemotherapy frequently causes constipation or diarrhea. In addition, the drugs used in this type of treatment reduce saliva production, causing a dry mouth with the risk of lesions and ulceration, known medically as mucositis. The resulting different levels of infection can be very painful for patients, preventing them from eating properly. To reduce this risk, it is essential to keep the mouth clean and moist by drinking lots of fluids and consuming food that is refreshing to the palate with a high nutritional content, as cancer therapy leads to a great loss of proteins, carbohydrates, and vitamins, etc., which is demonstrated in the reduction in patients' muscular mass and considerable weight loss.

Taking into account all the afore-mentioned, the proposed initiative significantly contributes to counteracting the negative effects of chemotherapy.

### CURRENT STATE OF INDUSTRIAL PROPERTY

A search was made in the main invention patent offices in the world and the following documents relevant to the present invention were found:

The US invention patent U.S. Pat. No. 5,922,375 (1999) Probiotic bifidobacterium strain, describes a probiotic bifidobacterium strain isolate that is incorporated into liquid foods, animal feeds, and/or dietary supplements. It is given as a healthy bacterium to humans and other mammals. This bacterium helps newborns to produce acetic acid and protective lactic acid, as well as antimicrobials and vitamins. The bacterium can also be used to reseed bacteria levels lost through diarrhea, chemotherapy, advancing age, antibiotics, or other causes. The main difference between this technology and the one seeking protection lies in the strain that is being claimed and its use, in addition to the fact that protection is aimed at the incorporation of food.

At the world patent office, invention patent WO0182711 (2001), Lactic acid food products, refers to food products that contain the probiotic strain Lactobacillus acidophilus and soy milk. The foods mentioned include a variety of dairy products such as sour cream, cottage cheese, desserts and ice cream. The difference between this technology and the initiative seeking protection lies mainly in the formulation used for the preparation of the said foodstuffs and the use given to the formulation.

Chinese invention patent CN1552229 (2004), Health-care ice cream powder containing active lactic acid bacteria, protects an ice cream dessert to safeguard health that is in powder form and contains active lactobacilli, prepared from sugar, powdered milk, vegetable butter, maltodextrine, sucrose ester, gum, CMC, essence, and coloring, etc. This product can maintain an ecological microenvironment in the human body and promotes the decomposition and absorption of nutrients. It is different from the technology seeking protection with regard to the formulation and use given to this functional food.

The Swedish invention patent SE526711 (2005), New strain of bifidobacterium with the ability to survive in the intestinal tract and produce glutamine and arginine in vivo, useful for preparing medicine for the treatment of intensive care patients with intestinal failure, protects a bifidobacterium strain with the ability to survive in the intestinal tract and produce glutamine in vivo. This patent claims a bifidobacterium strain, a composition that includes the strain with a carrier and a medicine for the treatment of patients with multiorgan dysfunction and intestinal damage requiring intensive care. This medicine can be used for prophylaxis in patients on chemotherapy and for patients with inflammatory or post-operative problems. The initiative seeking protection is not a medicine and the strain used is different from the one claimed in the above invention patent.

The Russian invention patent RU2294647 (2007), Ice cream with functional characteristics, protects an ice cream consisting of milk, cream, sugar, stabilizers, vitamin complexes and a bacteria concentrate containing Bifidobacterium lognum, Lactobacillus acidophilus and Propionibacterium shermanii. The technology seeking protection is different from the above, both in its formulation and in the use given to the formulation.

In addition to the review of existing patents, a search was conducted for relevant documents or publications relating in any way to the technology seeking protection. Among the documents found there are a variety of publications that mention probiotic foods, such as: Survival of lactobacillus acidophilus and bifidobacterium bifidum in ice cream for use as a probiotic food (Sharareh Hekmat, 1992. Journal of Dairy Science 75: 1415-1422) and Survival of probiotic microoganisms lactobacillus acidophilus and bifidobacterium lactis in whipped ice cream (Alejandro Corrales, June 2007. Revista Chilena de Nutrición Vol. 34 N° 2, pp. 157-163). These articles compare two probiotic strains, measuring parameters such as survival, changes in the organoleptic properties of the product, etc. Other publications were also found relating to the utility of certain foods for people suffering from colitis or immunological problems, but none of these foods totally reproduces the initiative proposed here.

### DESCRIPTION OF THE INVENTION

The present invention corresponds to a functional food for cancer patients who are immunocompromised because of the treatment they have been given. This food is prepared from a probiotic formulation whose active principle includes viable probiotic strains that produce lactic acid, Lactobacillus spp, preferably but not exclusively, the strain DSM 22105, isolated from breast milk, conditioned to withstand temperatures between −30 and −40° C., maintaining its concentration in a range from 10⁶-10⁹ CFU/ml, for long periods of time of at least 120 days. This formulation makes possible the production of functional foods, preferably ice cream, in addition to jellos, desserts, juices and/or milk derivatives, which are administered orally to cancer patients, preferably on chemotherapy and radiotherapy treatment, where the said food favors weight increase in these patients and counteracts the side effects produced by these types of therapies, such as mucositis, mouth dryness, lesions and erosions in the digestive tract.

To create this product, first the DSM 22105, isolated from breast milk, was selected from a number of probiotic strains, because it is the microorganism that has shown the most promising results in comparison with other strains of the same origin. (Table 1)

TABLE 1

| | | | | | Perox- |
|---|---|---|---|---|---|
| | | | | | ide |
| | | | | | Pro- |
| | | Hydrophobicity (%) | | | duc- |
| Strains | Origin | Hexadecane | Toluene | Xylene | tion |
| LPM O1 | Breast | 85 | 65.9 | 64.7 | + |
| L. plantarum | milk | | | | |
| LPM B₄ | Breast | 74 | 80.6 | 69.9 | +++ |
| L. plantarum | milk | | | | |
| LPM AK1 | Breast | 80.6 | 86.7 | 79.2 | +++ |
| L. plantarum | milk | | | | |
| LPM AR2 | Breast | 84 | 88 | 91 | — |
| Lactobacillus spp. | milk | | | | |
| LPM P1 | Breast | 66.5 | 72.6 | 68.2 | ++ |
| L. salivarius | milk | | | | |

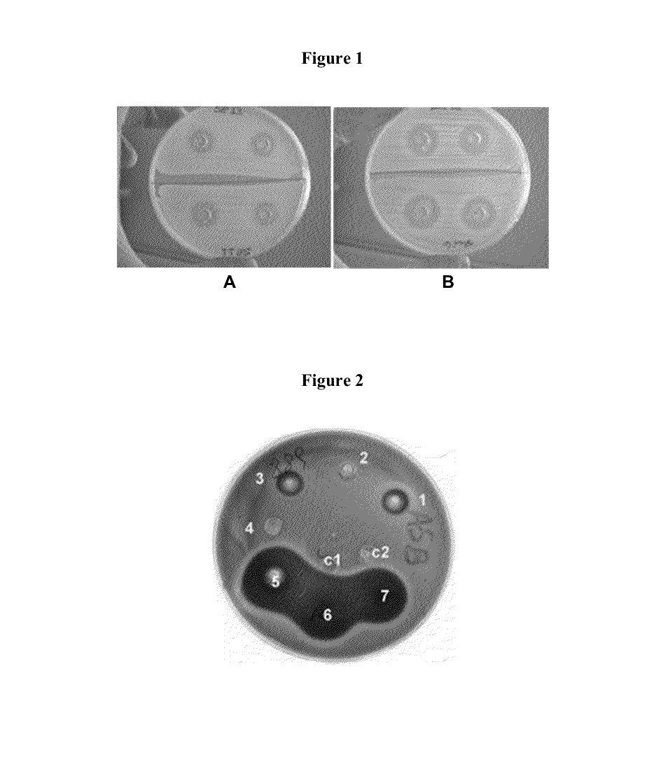

Studies have shown that, in the case of gastrointestinal pathologies, probiotic bacteria can be used both for therapeutic and preventive purposes. If these bacteria are added to certain foods in different ways, they are effective in the prevention and treatment of various illnesses. Some studies show that children given probiotic supplements present an increase in immunoglobulins, which decreases the intensity and duration of diarrhea produced by rotavirus. Probiotics are even able to inhibit the growth of hospital pathogenic bacteria, for which reason inhibition tests were carried out, (FIG. 1, A Salmonella enteritidis, B Shigella spp) in which the ability of strain LPM O1 (DSM 22105) isolated from breast milk to inhibit hospital pathogens and Campylobacter jejuni was evaluated (FIG. 2) in interaction trials in a cell culture dish.

In addition to the hospital pathogen inhibition tests, immunization tests with ovalbumin (OVA) were carried out on mice fed on the DSM 22105 strain, to demonstrate the beneficial effects of this strain. To this purpose, the mice were organized into three experimental groups and two control groups, in the following way:

Group 1 (preventive): the mice were fed for 7 days with the DSM 22105 strain with a dose of 10⁷ in serum glucose and then they were immunized with OVA antigen.

Group 2 (preventive): the mice were fed for 7 days with the DSM 22105 strain with a dose of 10⁷ in serum glucose, then they were immunocompromised with dexamethasone (4 doses, one every two days) and were immunized with OVA antigen from day 7.

Group 3 (control): the mice were exposed to OVA antigen.

Group 4 (control): the mice were immunocompromised with dexamethasone and immunized with OVA antigen.

Group 5 (curative): the mice were immunocompromised with dexamethasone, immunized with OVA antigen, and then fed for 7 days on the DSM 22105 strain with a dose of 10⁷ in serum glucose.

Subsequently, blood samples were taken from each experimental group at 0, 7, 14, 21, and 28 days from the first immunization, and then at 7, 14, 21 and 28 days after the second immunization. Table 2 shows the title of antibodies obtained.

TABLE 2

| Time (days) | Group 1 | Group 2 | Group 3 | Group 4 | Group 5 |
|---|---|---|---|---|---|
| 0 | — | — | — | — | — |
| 7 | — | — | — | — | — |
| 14 | 1/300 | 1/600 | 1/300 | 1/300 | 1/1000 |
| 21 | 1/600 | 1/600 | 1/600 | 1/600 | 1/1000 |
| 28 | 1/600 | 1/600 | 1/600 | 1/800 | 1/1000 |
| 7 (2° inm) | 1/6000 | 1/12000 | 1/6000 | 1/6000 | 1/12000 |
| 14 (2° inm) | 1/6000 | 1/12000 | 1/6000 | 1/6000 | 1/12000 |
| 21 (2° inm) | 1/6000 | 1/12000 | 1/6000 | 1/6000 | 1/12000 |
| 28 (2° inm) | 1/3000 | 1/6000 | 1/3000 | 1/6000 | 1/6000 |

As can be observed in Table 2, the results obtained by administering the DSM 22105 strain orally show that at 14 days all groups presented an increase in the title of antibodies, but the curative group presented the greatest increase. The response was similar at the different times studied. Nevertheless, after the second immunization, a much more significant antibody title increase was observed, especially in the preventive groups (immunocompromised) and the curative group, which had the highest title of antibodies, revealing that the DSM 22105 strain significantly reactivates the humoral immune response.

Once these properties have been determined, the DSM 22105 strain is conditioned and its ability to withstand extreme temperatures increased in order to finally produce a functional food, preferably a specially prepared ice cream.

To condition the strains to withstand low temperatures, certain procedures were carried out and elements produced that allow the DSM 22105 strain to acquire this property during the culture process and subsequent handling.

First, the culture of this lactic bacterium was optimized. To this purpose, a culture medium was used containing hydrolyzed whey permeate, which was the main carbon source of the medium and which, in this particular case, acted as a cryoprotectant, with additives and magnesium and manganese salts.

The addition of nonionic surfactants derived from sorbitan, such as polyoxyethylene (20) sorbitan monooleate, modified the permeability of the bacteria cell membrane, contributing to its survival during the frozen stage, because this increased the proportion of unsaturated fatty acids in the cell membrane. This fact is important as the strain in question was cultured in a medium containing polyoxyethylene (20) sorbitan monooleate and the biomass obtained was not washed, so that it contained a significant quantity of undigested nutrients, among which was the compound described above.

Another important factor is the physiological state of the bacteria. Bacteria in the stationary phase are more resistant to freezing or drying by sublimation (liophylization) than bacteria in the exponential phase. The culture was therefore harvested at 12 hours, which corresponded to the stationary phase of the DSM 22105 strain.

The addition of cryoprotectant agents, in addition to whey permeate, sorbitol, monosodium glutamate, and glycerol, among others, attenuates damage to the membrane during the freezing of the bacterial strains. At the same time, sucrose, a main component of ice cream, also has a protecting effect through its interaction with membrane proteins, thus reducing cell damage.

The nature of the additives is critical during the storage phase, since they directly influence the viability of the bacteria, but at the same time they should not alter the quality of the food product. To conclude, both the culture medium and the additives in the probiotic ice cream formulation contain cryoprotectant agents, which explains the maintenance of the viability of this product during storage time.

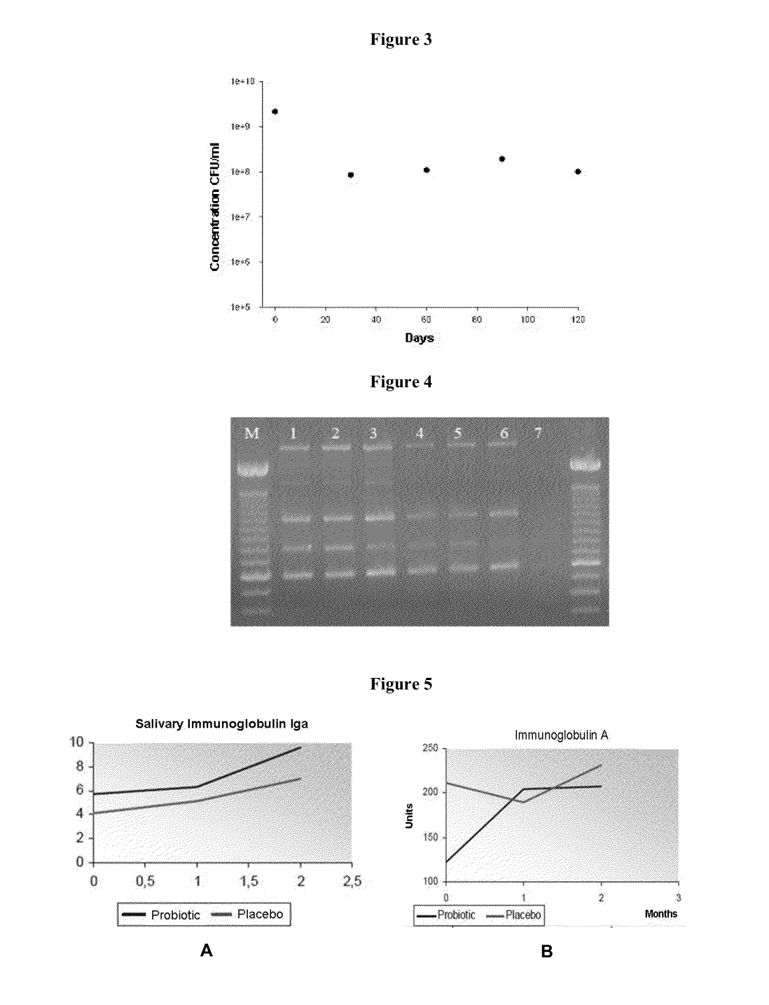

Once the conditions in which the DSM 22105 strain maintains its properties at low temperatures had been determined, a consignment of specially prepared ice cream was produced. This ice cream kept its organoleptic properties and possessed excellent stability indices for probiotic mass, attaining 1×10⁸ CFU/ml at the end of the monitoring process. This was determined from the samples taken from each container of prepared ice cream, all of which were homogenized and planted in different culture media to evaluate the viability (CFU/ml) and probiotic properties. FIG. 3 shows that the DSM 22105 strain incorporated into the special-purpose ice cream maintained high probiotic concentrations of around 1×10⁸ CFU/ml for at least 120 days from the day of preparation.

With regard to the biochemical characteristics of the DSM 22105 strain, in Table 3 it can be observed that these characteristics were maintained with respect to the original strain, which shows that the conditioning is effective and that the strain is able to withstand low temperatures without losing its properties.

TABLE 3

| | | | | | | | | | Peroxide |
|---|---|---|---|---|---|---|---|---|---|
| Date | Arginine | Esculin | Nitrate | Indole | Gluconate | Gibson | T ° 15° C. | T ° 45° C. | production |
| Initial | Negative | Positive | Negative | Negative | Negative | Negative | Negative | Positive | Negative |
| 30 days | Negative | Positive | Negative | Negative | Negative | Negative | Negative | Positive | Negative |
| 60 days | Negative | Positive | Negative | Negative | Negative | Negative | Negative | Positive | Negative |
| 78 days | Negative | Positive | Negative | Negative | Negativee | Negative | Negative | Positive | Negative |

Table 4 shows the evolution of the probiotic property of hydrophobicity of the strain in the ice cream. It can be seen that the property was maintained with respect to the original.

TABLE 4

| TIME | Index | XYLENE | TOLUENE | HEXADECANE |
|---|---|---|---|---|
| Initial | High | 85.6% | 80.9% | 83.6% |
| 30 days | High | 86.8% | 85.3% | 78.2% |
| 60 days | High | 87.1% | 79.8% | 81.1% |
| 78 days | High | 84.7% | 86.8% | 85.4% |

FIG. 4 shows the polymorphic profile obtained by RAPD-PCR of the DSM 22105 strain and the colonies isolated from the ice cream samples analyzed at 30 and 70 days: track 1 corresponds to the DSM 22105 strain (extraction kit); track 2 to ice cream colony at 30 days (extraction kit); track 3 to ice cream colony at 70 days (extraction kit); track 4 to the DSM 22105 strain (enzymatic extraction); track 5 to ice cream colony at 30 days (enzymatic extraction); track 6 to ice cream colony at 70 days (enzymatic extraction); track 7 to negative control and molecular weight (M) marker. In this figure it can be observed that the profiles show the same banding, the difference in intensity being produced by the type of extraction carried out, as bands 1, 2, and 3 were produced with a commercial extraction kit and bands 4, 5 and 6 were produced through enzymatic extraction.

From these analyses it can be concluded that the probiotic strain DSM 22105 incorporated into the specially prepared ice cream maintained the probiotic concentrations throughout the monitoring process. The biochemical tests on the strains isolated from the ice cream containers maintained the same profile as the incorporated probiotic strain. In addition, it can be observed that the probiotic properties of the strains isolated from the preparations were maintained with respect to the original, and the organoleptic properties of the ice cream—color, flavor and texture—were maintained without variation during the whole monitoring process.

Once these parameters had been measured, tests were conducted on patients on chemotherapy, this being the objective of the production of probiotic ice cream. Five voluntary cancer patients, with colon cancer, breast cancer and ovarian cancer, among others, all on chemotherapy, were recruited in addition to six healthy volunteers. A randomized double blind trial was designed, in which, by drawing lots, the volunteers selected the preparation according to its color, orange or green. The orange preparations contained only ice cream and the green preparations contained the probiotic strain. This identification was carried out at the moment of analyzing the results, not before, and the contents of the preparations were known only to the person in charge of the production process. The trial lasted 60 days, sensitization procedures being conducted beforehand, so that volunteers began the process with informed consent.

In the case of the healthy volunteers, a summary of the individual evolution of the participants with respect to saliva and serum immunoglobulin A is presented in Tables 5 and 6, as averages 3 volunteers with ice cream containing probiotics and 3 volunteers with a placebo, at 0, 30 and 60 days of ice cream consumption, and in FIGS. 5A and B. Although the results obtained are within normal ranges, a more marked rise in serum IgA levels was observed in patients who consumed probiotic ice cream than in those who consumed placebos.

TABLE 5
Serum Immunoglobulin A (Average)

| | 0 | 30 days | 60 days |
|---|---|---|---|
| Probiotic | 122 | 204 | 208 |
| Placebo | 211 | 190 | 232 |

TABLE 6

| Salivary IgA (Average) | 0 | 30 days | 60 days |
|---|---|---|---|
| Probiotic | 5.7 | 6.3 | 9.6 |
| Placebo | 4.1 | 5.1 | 7.0 |

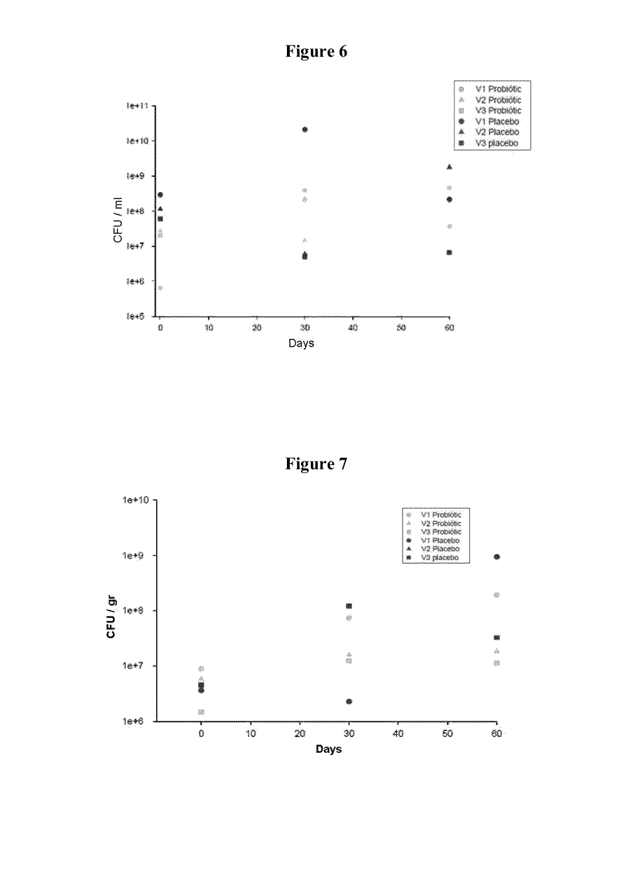

Then a microbiological recount was conducted in stools from healthy volunteers. FIG. 6 gives a graphic presentation of the lactic acid flora concentrations obtained from healthy volunteers analyzed up to 60 days. It can be observed that these volunteers had a basal lactobacillus concentration of approximately 10⁷-10⁹ CFU/ml. At 30 days differences were observed between the two groups of volunteers. In the probiotic group all the volunteers increased their concentration significantly, while the placebo group showed a decrease. At the end of the 60-day trial, the probiotic group presented recounts similar to those obtained at 30 days, while the placebo group showed an increased concentration in comparison with the 30-day recounts.

In the cancer volunteers, it was observed that the IgA parameters were more stable in the patients who consumed the preparation with probiotics in comparison with the patients who took the placebo (Table 7, as averages 3 volunteers with probiotic ice cream and 2 volunteers with the placebo, at 0, 30 and 60 days of ice cream consumption). A progressive, sustained increase over time was observed in the people on probiotic ice cream, in contrast to the volunteers on the placebo, whose evolution was more erratic. Although an increase in salivary IgA was observed at day 60, this increase was not statistically significant (ANOVA p=0.790123).

TABLE 7
Salivary Immunoglobulin IgA (Average)

| | 0 | 30 days | 60 days |
|---|---|---|---|
| Probiotic | 4.5 | 6.7 | 8.3 |
| Placebo | 5.4 | 3.4 | 1.7 |

With respect to IgA evolution, (Table 8, as averages 3 volunteers on probiotic ice cream and 2 volunteers on placebo, at 0, 30 and 60 days of ice cream consumption), behavior was very erratic in placebo users, in contrast to probiotic ice cream users, whose evolution was more stable. Although a difference was observed, this difference is not statistically significant (ANOVA p=0.149651).

TABLE 8
Serum Immunoglobulin A (Average)

| | 0 | 30 days | 60 days |
|---|---|---|---|
| Probiotic | 178 | 186 | 157 |
| Placebo | 147 | 127 | 256 |

One of the most important parameters to consider, and one that makes this initiative more innovative, is related to the weight variations in patients on chemotherapy. The volunteers who ingested the preparation with the DSM 22105 strain showed marked body weight recovery, increasing their weight by 2 to 6 kg, preferably 3 kg per month (Table 9). The weight difference between the beginning and after one month of treatment was statistically significant according to the t-test for paired data (p=0.00492623). The ANOVA test for correlated samples shows that the weighted mean difference at the beginning, and at 30 and 60 days was significant p<0.03381 and Tukey's HSD test shows that the pair that indicated significant weighted mean difference was between 1 and 2 and between 1 and 3 (beginning, 30 days and 60 days).

TABLE 9

| | | WEIGHT | WEIGHT |
|---|---|---|---|
| | INITIAL WEIGHT | 30 DAYS | 60 DAYS |
| TREATMENT | Kg | Kg | Kg |
| PROBIOTIC (patient 1) | 68 | 72 | 72 |
| PROBIOTIC (patient 2) | 84 | 87 | 88 |
| PROBIOTIC (patient 3) | 67 | 70 | 71 |

Table 10 presents the results of patients treated with the placebo, showing that the weighted mean difference between the beginning and end of treatment was not significant in these patients, thus confirming that the probiotic strain DSM 22105 benefits cancer patients by enabling them to gain weight.

TABLE 10

| | INITIAL WEIGHT | WEIGHT | WEIGHT |
|---|---|---|---|
| TREATMENT | Kg | 30 DAYS | 60 DAYS |
| PLACEBO (patient 4) | 71 | 72 | 70 |
| PLACEBO (patient 5) | 64 | 64 | 63 |

The microbiological recount in the stools of cancer volunteers (FIG. 7) shows the lactic acid bacteria concentrations obtained from volunteers on chemotherapy, analyzed up to 60 days. At the beginning of the trial, all the volunteers presented a low concentration of basal lactobacilli from 10⁶-10⁷ CFU per gram of feces, which at 60 days had increased to levels around 10⁸ CFU per gram of feces. These data show that there is a clear recovery in the basal flora in the intestinal bacterial population

With regard to the product itself, the organoleptic properties of the ice cream—color, flavor and texture—remained the same during the monitoring process and were greatly appreciated by the participants. This reaffirms the fact that the strain incorporated into the ice cream does not change its biochemical properties and it also confirms the recovery of the sense of taste and the perception of flavor of cancer patients treated with the ice cream.

It is also important to consider the side effects of the consumption of the preparation. Not a single one of the patients in the two groups that ate the probiotic preparation had diarrhea after ingesting the product. Nor were there any other adverse reactions, such as mucositis, nausea or vomiting, like those described in most patients on chemotherapy and radiotherapy.

The preparation is conceptualized as a dessert. It was described as having a smooth texture and delicious flavor. The same appreciation was shown by both cancer volunteers and healthy volunteers. Even more importantly, for the cancer volunteers these three factors played a motivating role in maintaining regular consumption, despite the different states through which the body passed as a result of chemotherapy: an intense burning sensation in the upper digestive tract, general weakness, nausea, and constant headaches, as well as feelings of negativity. In addition, participants said that the probiotic ice cream was a nutritional supplement, since they did not stop eating it even on days when they were physically unwell. In fact, the ice cream was sometimes the only food they ate.

### APPLICATION EXAMPLES

### Example 1

### Activation and Evaluation of the Probiotic Strain

1.1 Preparation of DSM 22105. The strain was activated from a strain bank; after its activation, the bacteria were centrifuged, washed with physiological solution and adjusted to an optical density of OD₅₄₀ 0.6, then planted using torula on an MRS agar surface, and incubated for 24 hours at 25° C.

1.2 Preparation of hospital pathogens and Campylobacter. Jejuni. The enteropathogenic strains were activated and brought to a McFarland concentration of 0.5, then planted on a trypticase agar plate, in the case of the hospital pathogens, and on brucella agar with 5% horse blood, in the case of C. jejuni. 20 strains of Shigella spp., 20 strains of Escherichia coli, 30 strains of Salmonella spp., and 3 strains of C. jejuni isolated from patients with Diarrheic Syndrome were used.

For the inhibition trial conducted on trypticase agar plates for the development of the pathogenic strain, the lactic strain was inoculated on a microplate and incubation was carried out at 37° C. for 12 and 24 hrs.

1.3 Interaction trial with Campylobacter Jejuni. This trial was conducted using the microplate interaction technique with modifications, plus Campbell's method. For the microplate interaction technique, the DSM 22105 strain was subjected to treatment using 5 conditions:
Peroxide inhibition
Lactic acid neutralization
Peroxide inhibition and lactic acid neutralization
Supernatant without bacteria
Whole culture at an approximate concentration of 10⁹ CFU/ml.

For the Campbell's trial, pieces of MRS agar in which the lactic strain had already been grown were taken and they were deposited on the brucella agar surface that had been recently planted with C. jejuni strains; a disc with the lactic strain was placed in contact with the pathogen and another disc was placed with the surface in contact with the pathogenic strain. After the trial it was incubated in a microaerophilic environment at 37° C. for 48 hours.

### Example 2

### Culture and Conditioning of the LPM O1 Strain

2.1 Preparation of Culture Medium.

Hydrolysis of serum permeate: The amount of enzyme needed (lactase or β-galactosidase) was added to serum permeate dissolved in distilled water and incubated for 1 to 4 hours at 40° C., under constant agitation.

Addition of nutrients: After hydrolysis had taken place, the remaining components from the culture medium were added, and it was adjusted to pH=6.0 and autoclaved for future use.

| Culture medium components | Quantity (g/L) |
|---|---|
| Lactoserum permeate | between 10-70; preferably between 30-50 |
| Enzyme (β-galactosidase) | between 0.1-4; preferably between 0.25-2 |
| Casein peptone | between 1-10; preferably between 4-7 |
| Yeast extract | between 1-8; preferably between 2-5 |
| Dihydrogen phosphate potassium | between 0.5-7; preferably between 1-4 |
| Polyoxyethylene (20) sorbitan monooleate | between 0.1-3; preferably between 0.5-1.5 |
| Magnesium sulfate | between 0.005-0.1; preferably between 0.01-0.03 |
| Manganese sulfate | between 0.05-2, preferably between 0.2-0.5 |

2.2 Culture of the DSM 22105 strain. After the DSM 22105 strain had been activated at 37° C., in consecutive picks, the first for 24 h and the second for 12 h, the concentration was adjusted to an OD of 0.5 (625 nm), in order to then inoculate the amount needed in the fermentation with the lactoserum permeated medium at 37° C., ensuring an initial concentration of bacteria of 10⁵ CFU/ml. This was incubated at 37° C. for approximately 12 hours.

2.3 Biomass harvesting. Once the culture time was over, the broth was centrifuged with the biomass at 8000 rpm for 20 min at 15° C. After the sample was centrifuged, the supernatant was discarded and the decanted biomass stored for later use at 4° C.

2.4 Mixture. The biomass obtained from the culture was resuspended in whole milk (part of the ice cream composition) and the suspension was homogenized in an orbital agitator, to which was added the remaining liquid ice cream mixture (previously pasteurized). This was stored at 4° C., ready for the freezing stage of the probiotic ice cream.

### Example 3

### Preparation of the Dairy Ice Cream with a Probiotic

Ingredients

50 gr. ice cream base

40 gr. fat preparation

240 gr. sugar

1 lt. milk

50 gr. dairy cream

1 gr. lactic bacteria with 10⁹ CFU

65 gr. flavoring

3.1 Ingredient mixing: A homogenous mixture of the different ingredients was obtained by heating the liquids (this is normally done using temperatures from 30-40° C.) and then adding the powder base mixed with the stabilizer and emulsifier.

3.2 Pasteurization: A product free from viable pathogenic bacteria was obtained and the storage quality of the product was improved. Heating the mixture had the effect of dissolving the sugars, stabilizers and emulsifiers, while at the same time liquefying the fats. The recommended temperature for the continuous process is 80° C. for 25 seconds and 72° C. for 30 minutes for discontinuous pasteurization.

3.3 Homogenization: This stage is designed to obtain a fat globule of uniform size in emulsion, distribute the emulsifiers and milk proteins on the surface of the fat globule (protector colloids), improve the mixture and incorporate air, produce a smooth texture, and improve melting.

3.4 Cooling: Once the mixture was homogenized, it was cooled to 4° C. to allow it to mature. This process can take from 2 to 4 hours.

3.5 Incorporation of probiotic lactic bacteria: Taking advantage of the coldness of the mixture, the lactic strain was incorporated by gentle mixing; making sure that a concentration of 10⁹ CFU/ml was uniform throughout the contents.

3.6 Freezing: The freezing and beating of the mixture transform it from a liquid state to a semi-solid state. The air content incorporated depends on the mixing speed in the freezer. The exit temperature from the freezer fluctuates around −5° C., and at this temperature practically 50% of the water in the mixture is in a solid state.

3.7 Hardening and Storage: Once the product comes out of the freezer in containers, it stabilizes rapidly, and most of the water that is still in liquid form freezes when the temperature is reduced to between −30° C. and −40° C., which hardens the ice cream.

After the hardening process the ice cream can be stored in a freezer at −20° C. until it is commercialized, at which point it is necessary to use appropriate cold channels in order to avoid undesirable changes in texture caused by fluctuations in storage temperature.

Finally, the ice cream must have a concentration of probiotic lactic bacteria of 10⁸ CFU/g.

3.8 Stability of the product. The specially prepared ice cream was produced in 200 ml. containers. From each container approximately 5 ml was extracted, homogenized and planted in different culture media for an evaluation of the viability (CFU/ml) and probiotic properties. The plates were incubated from 24 to 48 hours at 37° C. The ice cream was stored in a conventional freezer at −20° C.

### Example 4

### Clinical Trial

4.1 Inclusion Criterion. Five volunteer cancer patients with colon, breast and ovarian cancer, among others, who were in chemotherapy cycles, were recruited, together with six healthy volunteers.

4.2 Recruitment. A randomized double blind trial was designed, in which, by drawing lots, the volunteers selected an orange or green preparation. The orange preparations contained only the ice cream and the green preparations contained the probiotic strain. This identification was only made at the moment of analyzing the results and the contents of the preparations were only known to the person in charge of the production process. The trial lasted 60 days and was preceded by a sensitization process, so that the volunteers began participation with informed consent before they drew lots for the preparation to be ingested.

4.3 Dosification. The clinical trial considered the consumption of the probiotic strain DSM 22105 at a probiotic concentration of 10⁷-10⁹ CFU/ml in each preparation, which was eaten for a month in a dose of 100 ml of ice cream daily. These preparations were stored in a freezer at −20° C. Each participant was given educational material with instructions to follow, including a chart on which to record symptoms.

4.4 Monitoring of the preparations. The following protocol was used to determine the viability of the preparation. Ice cream samples were planted serially to determine the number of colony forming units (CFU/ml of ice cream) on Rogosa and MRS agar plates. The results were analyzed after 48 hours of incubation at 37° C. under microaerophilic conditions.

4.5 Clinical evaluation. At 0, 30 and 60 days, every participant was given a check-up, including a hemogram, and analyses of ESR, C-reactive protein, salivary IgA, the C3 factor of the compliment system, and of a stool sample. In addition, cancer patients were asked to chart their diarrhea episodes on a daily basis, according to the classification provided by the World Health Organization (WHO):

Grade 0=No diarrhea.

Grade 1=Increase of 2 to 3 bowel movements/day over basal.

Grade 2=Increase of 4-6 bowel movements/day or nocturnal bowel movements, or moderate pains.

Grade 3=Increase of 7-9 bowel movements/day, incontinence or strong pains.

Grade 4=Increase of more than 10 bowel movements/day, visible bloody diarrhea or need for parenteral support.

4.6 Stool analysis. Three stool samples were taken from each participant, the first before the ice cream was ingested (TO), and then at 30 days (T30) and 60 days (T60) after consumption. The stool samples were transported in sterile containers in a cold chain. The samples were planted in MRS selective medium and incubated at 37° C. under microaerophilic conditions (5% CO₂) for 24-48 hours. The methodology described made it possible to determine the presence and concentration of lactic bacteria present in volunteers' stools. Colonies with different morphology were selected from the plates with positive growth for incubation in MRS broth for 24 h at 37° C. Gram staining and a catalase test were carried out on the tubes with positive growth, and the catalase-negative and Gram-positive strains with bacillary or coccobacillary morphology were selected and kept in milk supplemented by yeast extract at −20° C., for molecular studies.

1. A probiotic functional food, suitable for patients on therapies such as chemotherapy and/or radiotherapy, its formulation including:
as active principle, a biomass of viable probiotic strains that produce lactic acid, Lactobacillus spp, preferably but not exclusively the strain DSM 22105, which is conditioned to withstand temperatures from −30 to −40° C., and
cryoprotectant agents.

2. A probiotic functional food, according to claim 1, wherein the strain DSM 22105 is isolated from breast milk.

3. A probiotic functional food, according to claim 1, wherein the strain DSM 22105 increases the title of antibodies and reactivates the humoral immune response in immunocompromised individuals.

4. A probiotic functional food, according to claim 1, wherein the biomass of DSM 22105 is found in a concentration range of 106-109 CFU/ml, preferably 108 CFU/ml, for at least 120 days.

5. A probiotic functional food, according to claim 1, wherein the cryoprotectant agents include:
lactoserum permeate; at a concentration of 10-70 g/L, preferably 30-50 g/L and,
sorbitan derivatives, preferably polyoxyethylene (20) sorbitan monooleate, at a concentration of 0.1-3 g/L, preferably 0.5-1.5 g/L.

6. A probiotic functional food, according to claim 1, wherein the said food includes jellos, desserts, juices and/or milk derivatives, preferably ice cream.

7. A method of using a probiotic functional food suitable for patients on therapies such as chemotherapy and/or radiotherapy, wherein the said food includes the strain DSM 22105, isolated from breast milk and used to inhibit the growth of hospital pathogens, preferably Salmonella enteritidis, Shigella spp and Campylobacter jejuni.

8. A method of using a probiotic functional food suitable for patients on aggressive therapies, wherein the said food includes the strain DSM 22105, isolated from breast milk and used preferably in immunocompromised individuals, preferably cancer patients on chemotherapy and/or radiotherapy.

9. A method of using a probiotic functional food, according to claim 8, wherein it is used for weight gain in cancer patients on radiotherapy and/or chemotherapy, between 2 and 6 kg, preferably 3 kg per month.

10. A method of using a probiotic functional food, according to claim 8, wherein it counteracts the side effects produced by chemotherapy, preferably mucositis, mouth dryness, and lesions and erosions in the digestive tract.

11. A method of using a probiotic functional food, according to claim 8, wherein it is used for the recovery of the sense of taste and the perception of flavor in cancer patients on chemotherapy.
